(19) United States
(12) Patent Application Publication
(10) Pub. No.: US 2014/0094434 A1
(43) Pub. Date: Apr. 3, 2014

(54) AMINO ACID COMPOSITION

(71) Applicant: AJINOMOTO CO., INC, Chuo-ku, Tokyo (JP)

(72) Inventors: Yuri HIRABAYASHI, Kawasaki-shi (JP); Chie FURUTA, Kawasaki-shi (JP); Yoshihito NOGUSA, Kawasaki-shi (JP); Katsuya SUZUKI, Kawasaki-shi (JP); Hisamine KOBAYASHI, Kawasaki-shi (JP)

(73) Assignee: AJINOMOTO CO., INC., Chuo-ku, Tokyo (JP)

(21) Appl. No.: 14/098,691

(22) Filed: Dec. 6, 2013

Related U.S. Application Data
(63) Continuation of application No. PCT/JP2012/064721, filed on Jun. 7, 2012.

(30) Foreign Application Priority Data
Jun. 7, 2011 (JP) 2011-127747
Oct. 28, 2011 (JP) 2011-237322

(51) Int. Cl. A61K 31/721; A61K 31/401; A61K 31/198

(52) U.S. Cl. CPC A61K 31/721; A61K 31/198; A61K 31/401
USPC 514/58; 562/576; 548/532; 514/561; 514/423; 562/553

(57) ABSTRACT

Amino acid compositions containing a carbohydrate and any one or more kinds of alanine, proline and glycine as amino acid can suppress a rapid increase in the blood glucose level immediately after ingestion and a decrease in the blood glucose level due to prolonged exercise, and can improve exercise performance. In addition, the present invention provides inhibitors of an increase in the blood glucose level immediately after carbohydrate ingestion, an inhibitor of a decrease in the blood glucose level due to a prolonged exercise, and an exercise performance improver. Therefore, the present invention provides an energy supplement useful for those who exercise (particularly prolonged exercise).

### CROSS REFERENCES TO RELATED APPLICATIONS

This application is a continuation of International Patent Application No. PCT/JP2012/064721, filed on Jun. 7, 2012, and claims priority to Japanese Patent Application No. 2011-127747, filed on Jun. 7, 2011, and Japanese Patent Application No. 2011-237322, filed on Oct. 28, 2011, all of which are incorporated herein by reference in their entireties.

### BACKGROUND OF THE INVENTION

1. Field of the Invention

The present invention relates to amino acid compositions, which contain a carbohydrate and an amino acid, more particularly, to an amino acid compositions used for improving exercise performance, which suppress an increase of blood glucose level in an initial stage of the composition ingestion, and suppress a decrease of blood glucose level during exercise for a long time. Furthermore, the present invention relates to inhibitors of an increase of blood glucose level immediately after carbohydrate ingestion, an inhibitor of a decrease of blood glucose level during exercise for a long time, and an exercise performance improver. The present invention also relates to foods and drinks, and pharmaceutical products, which contain such a composition.

2. Discussion of the Background

Generally, prolonged exercise gradually lowers exercise performance due to muscle fatigue and a decrease of blood glucose level. To prevent this phenomenon, an appropriate energy supplementation is important. However, appropriate drinking and eating during exercise is often difficult. Therefore, particularly for those who exercise for a long time, inter alia players (athletes) requested to maintain high exercise performance for a long time, appropriate energy supplementation before exercise is important.

Amino acids have been regularly used as supplement by general public and athletes, and are reported to provide various physiological actions. For example, Metabolism, 2004, 53(2): 241-246, which is incorporated herein by reference in its entirety, discloses that ingestion of proline suppresses an increase of blood glucose level after glucose ingestion in humans. In addition, Biochem. J., 1991, 273: 57-62, which is incorporated herein by reference in its entirety, discloses that glutamine, proline, alanine, asparagine, and histidine enhance the glycogen synthesis in isolated rat hepatocytes. However, these documents do not describe any effect of amino acid ingestion on exercise performance. Moreover, JP-A-2002-3372, which is incorporated herein by reference in its entirety, discloses that a composition containing branched chain amino acids (BCAA), arginine and glutamine provides an effect of improving hematopoiesis and nutritional status. However, JP-A-2002-3372 does not describe any changes in the blood glucose level or an influence on the exercise performance resulting from the ingestion of other amino acids.

### SUMMARY OF THE INVENTION

The source of energy ingested during exercise is based on carbohydrates. When carbohydrates alone are ingested for energy supplementation before exercise, the blood glucose level rapidly increases immediately after ingestion, possibly inducing hypoglycemia due to an increase in the insulin secretion and the like. When exercise takes a long time, moreover, problems occur in that the blood glucose level lowers and exercise performance cannot be maintained sufficiently.

Accordingly, it is one object of the present invention to provide novel amino acid compositions.

It is another object of the present invention to provide novel amino acid compositions which suppress a rapid increase of the blood glucose level immediately after ingestion.

It is another object of the present invention to provide novel amino acid compositions which suppress a decrease of blood glucose level due to exercise for a long time.

It is another object of the present invention to provide novel amino acid compositions which are able to improve exercise performance.

It is another object of the present invention to provide novel foods, drinks, and pharmaceutical products, which contain such a composition.

These and other objects, which will become apparent during the following detailed description, have been achieved by the inventors' discovery that ingestion of any one or more kinds of alanine, proline, and glycine simultaneously with carbohydrate ingestion as an energy source suppresses a rapid increase of blood glucose level immediately after ingestion and a decrease of blood glucose level due to prolonged exercise. Furthermore, they have found that ingestion of any one or more kinds of alanine, proline, and glycine when carbohydrate is ingested or carbohydrates in the body are used as an energy source without carbohydrate ingestion improves exercise performance as compared to an ingestion of isocaloric carbohydrate alone.

Accordingly, the present invention provides the following:

(1) An amino acid composition, comprising a carbohydrate and any one or more kinds of alanine, proline, and glycine as amino acid.

(2) The amino acid composition of (1), wherein a total concentration of any one or more kinds of alanine, proline, and glycine is 2 wt % to 50 wt %, based on the solid content of the composition.

(3) The amino acid composition of (1) or (2), wherein a total concentration of the carbohydrate is 10 wt % to 90 wt %, based on the solid content of the composition.

(4) The amino acid composition of any of (1)-(3), which is in the packaged form of a unit ingestion amount of 2.5 g to 15 g in total of any one or more kinds of alanine, proline, and glycine.

(5) The amino acid composition of any of (1)-(4), comprising alanine, proline and/or glycine in combination as amino acid.

(6) The amino acid composition of (5), wherein alanine, proline and/or glycine are contained in a blending ratio of alanine:proline and/or glycine=1:0.01 to 1.

(7) The amino acid composition of any of (1)-(6), which is in the jelly or liquid form.

(8) The amino acid composition of any of (1)-(7), which suppresses an increase of blood glucose level immediately after ingestion as compared to ingestion of isocaloric carbohydrate.

(9) The amino acid composition of any of (1)-(7), which suppresses a decrease of blood glucose level due to prolonged exercise after ingestion as compared to isocaloric carbohydrate ingestion.

(10) The amino acid composition of any of (1)-(7), which is used for improving exercise performance.

(11) The amino acid composition of any of (1)-(10), which is free of other amino acids.

(12) An inhibitor of an increase of blood glucose level immediately after carbohydrate ingestion, comprising any one or more kinds of alanine, proline and glycine.

(13) An inhibitor of a decrease of the blood glucose level due to an exercise for a long time, comprising any one or more kinds of alanine, proline and glycine.

(14) An exercise performance improver comprising any one or more kinds of alanine, proline and glycine.

(15) A method of suppressing an increase of blood glucose level immediately after carbohydrate ingestion, comprising administering a composition containing any one or more kinds of alanine, proline and glycine.

(16) A method of suppressing a decrease of blood glucose level due to an exercise for a long time, comprising administering a composition containing any one or more kinds of alanine, proline and glycine.

(17) A method of improving exercise performance, comprising administering a composition containing any one or more kinds of alanine, proline and glycine.

The amino acid compositions provided by the present invention can suppress a rapid increase of blood glucose level immediately after ingestion and a decrease of blood glucose level due to prolonged exercise, and can improve exercise performance. In addition, the agents provided by the present invention can suppress a rapid increase of blood glucose level immediately after carbohydrate ingestion and a decrease of blood glucose level due to prolonged exercise, and can improve exercise performance. Therefore, an energy supplement useful for those who exercise (particularly prolonged exercise) can be provided.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows changes in the blood glucose level 15 minutes after oral administration of each sample solution described in Example 1.

FIG. 2 shows changes in the blood glucose after loading of 90 minutes exercise from 15 minutes after oral administration of each sample solution described in Example 1.

FIG. 3 shows changes in the blood glucose level 15 minutes after oral administration of each sample solution described in Example 2.

FIG. 4 shows changes in the blood glucose after loading of 90 minutes exercise from 15 minutes after oral administration of each sample solution described in Example 2.

FIG. 5 shows changes in the blood glucose level 15 minutes after oral administration of each sample solution described in Example 3.

FIG. 6 shows changes in the blood glucose after loading of 90 minutes exercise from 15 minutes after oral administration of each sample solution described in Example 3.

FIG. 7 shows a running time up to exhaustion in Example 4.

FIG. 8 shows cumulative spontaneous motor activity after loading of 90 minutes running exercise at 22 m/min in Example 5.

FIG. 9 shows an increase in the running time from that without administration in Example 6.

FIG. 10 shows changes in the blood glucose level from that before administration after loading of 60 minutes running exercise in Example 6.

FIG. 11 shows a liver glycogen content after loading of 60 min running exercise in Example 6.

FIG. 12 shows a swimming time up to exhaustion in Example 7.

FIG. 13 shows changes in the blood glucose level in Example 11.

FIG. 14 shows a swimming time up to exhaustion in Example 12.

FIG. 15 shows a running time up to exhaustion in Example 13.

FIG. 16 shows an increase in the running time after administration of distilled water in Example 13.

### DETAILED DESCRIPTION OF THE PREFERRED EMBODIMENTS

The amino acid composition of the present invention contains carbohydrate and any one or more kinds of alanine, proline, and glycine as amino acid.

The carbohydrates in the present invention may be any saccharide as long as it becomes a source of energy supplementation. Examples of the saccharide include, but are not limited to, monosaccharides (glucose, fructose, mannose, galactose, and the like), disaccharides (sucrose, lactose, maltose, trehalose, isomaltose, and the like), oligosaccharide and polysaccharides (starch, dextrin, glycogen, and the like), and the like. From the aspect of efficient energy supplementation, glucose, fructose, mannose, galactose, sucrose, lactose, maltose, trehalose, isomaltose, oligosaccharide, dextrin, and reduction dextrin are preferable as carbohydrates. Any one of the saccharides may be used as carbohydrate, and two or more kinds of saccharides may be used in combination.

Alanine (hereinafter sometimes to be abbreviated as Ala) contained in the amino acid composition of the present invention may be any of L-form, D-form, and DL-form.

Proline (hereinafter sometimes to be abbreviated as Pro) contained in the amino acid composition of the present invention may be any of L-form, D-form, and DL-form.

Moreover, the amino acid composition of the present invention may contain glycine (hereinafter sometimes to be abbreviated as Gly).

The amino acid contained in the amino acid composition of the present invention may be in the form of a physiologically acceptable salt. As the form of such salt, salt with an acid (acid addition salt), salt with a base (base addition salt) and the like can be mentioned. Examples of the acid that forms an acid addition salt include inorganic acids such as hydrogen chloride, hydrogen bromide, sulfuric acid, phosphoric acid, and the like; and organic acids such as acetic acid, lactic acid, citric acid, tartaric acid, maleic acid, fumaric acid, monomethylsulfuric acid, and the like. Examples of the base that forms a base addition salt include hydroxides or carbonates of a metal such as sodium, potassium, calcium, and the like, or an inorganic base such as ammonia and the like; and an organic base such as ethylenediamine, propylenediamine, ethanolamine, monoalkylethanolamine, dialkylethanolamine, diethanolamine, triethanolamine, and the like.

The amino acid composition of the present invention may be a composition containing any one or more kinds of alanine, proline, and glycine as amino acid. Alanine can be used for gluconeogenesis in the liver, and is known to play an important role in the supply of a sugar necessary in the body for continuous exercise for a long time.

On the other hand, a combined use of proline and/or glycine and alanine tends to suppress a decrease of blood glucose level due to prolonged exercise more than a use of alanine alone. Therefore, the amino acid composition of the present invention more preferably contains alanine, and proline and/or glycine in combination. In this case, the blending ratio of alanine, and proline and/or glycine is generally alanine:proline and/or glycine=1:0.01 to 1, preferably 1:0.05 to 0.5, more preferably 1:0.1 to 0.3, in a weight ratio. When the ratio of proline and/or glycine is higher than 1, carbohydrate may be browned, which is not preferable in appearance.

The total concentration of any one or more kinds of alanine, proline, and glycine in the amino acid composition of the present invention is generally 2 wt % to 50 wt %, preferably 3 wt % to 30 wt %, more preferably 5 wt % to 20 wt %, based on the solid content of the composition. When the total concentration of any one or more kinds from alanine, proline and glycine is less than 2 wt %, ingestion tends to become difficult since a single ingestion amount of the composition becomes high to ensure ingestion of a necessary amount of the amino acid. When it exceeds 50 wt %, even ingestion of a necessary amount of the amino acid tends to fail in sufficient energy supplementation, since the concentration of carbohydrates decreases.

The concentration of the above-mentioned solid content is calculated based on the total amount of any one or more kinds of alanine, proline and glycine added to the amino acid composition of the present invention wherein the total solid content of the composition is 100 wt %. Accordingly, when the amino acid composition of the present invention is a liquid, a jelly and the like, the aforementioned concentration does not vary depending on whether or not the blended starting materials are completely dissolved. In the present specification, the “solid content” refers to a solid part excluding volatile substances (for example, water and the like) from the composition and the like.

The total concentration of carbohydrates in the amino acid composition of the present invention can be appropriately determined according to the kind of the carbohydrates to be used, the form of the composition and the like. The total concentration of carbohydrates is generally, 10 wt % to 90 wt %, preferably 30 wt % to 90 wt %, more preferably 50 wt % to 90 wt %, based on the solid content of the composition. When the total concentration of carbohydrates is less than 10 wt %, sufficient energy supplementation tends to be unachievable even when the necessary amount of any one or more kinds of amino acids from alanine, proline, and glycine is ingested. In addition, when the total concentration of carbohydrates exceeds 90 wt %, ingestion of a necessary amount of the amino acid tends to be difficult when the necessary amount of carbohydrate is ingested, since the total concentration of the amino acid decreases.

The above-mentioned concentration as a solid content is calculated based on the total amount of carbohydrates mixed in the amino acid composition of the present invention, wherein the composition of the present invention is 100 wt %. Accordingly, when the amino acid composition of the present invention is a liquid, a jelly and the like, the aforementioned concentration does not vary depending on whether or not the blended starting materials are completely dissolved.

The blending (weight) ratio of the carbohydrate and any one or more kinds of amino acids from alanine, proline, and glycine in the amino acid composition of the present invention can be appropriately determined within the above-mentioned concentration range. Specifically, it can be determined within the range of carbohydrate:any one or more kinds of amino acid from alanine, proline, and glycine=1:0.0125 to 5, preferably 1:0.025 to 2, more preferably 1:0.05 to 1.

The amino acid composition of the present invention can be in the form of a package of a unit ingestion amount for one time or one meal. In such embodiment, the amount to be ingested once or per meal is determined in advance and packaged. Examples thereof include a form of a package of an amount to be ingested at one time using a container such as pack, bag, bottle, box in case of drinks, jelly, yogurt, gum, cookie, and the like, and a form of an individual package of an amount to be ingested at one time using pack, bag, and the like in case of granule, powder, slurry, and the like. Particularly, when the composition is a health food, functional food, food with nutrient function claims, food for specified health uses, and the like, for example, a form wherein the composition of the present invention is packed in a unit amount to be ingested once or per meal, a form wherein the composition of the present invention is suspended or dissolved to give a drink or a jelly, which is packaged in a pack etc. for a single consumption or ingestion and the like can be mentioned.

The above-mentioned ingestion amount for one time or one meal can contain any one or more kinds of amino acid from alanine, proline, and glycine in a total amount of 2.5 g to 15 g, preferably 3 g to 12 g, more preferably 4 g to 10 g. In this way, ingestion of one time or one meal unit ingestion amount enables convenient ingestion of the necessary amount of amino acid together with carbohydrates.

The above-mentioned one time or one meal ingestion amount can contain 1 g to 100 g, preferably 5 g to 80 g, more preferably 10 g to 60 g, of carbohydrates in total. In this way, ingestion of one time or one meal unit ingestion amount enables convenient supplementation of sufficient energy.

The form of the amino acid composition of the present invention may be liquid (drinks and the like), jelly (jelly, gel, jelly drinks, and the like), milky (milk, milk beverage, yogurt, and the like), solid (gum, powdered, granular, sheet, capsule, tablet, candy bar, cookies, and the like), and the like. From the aspect of easy ingestion, jelly or liquid is preferable.

When the form of the amino acid composition of the present invention is a jelly or a liquid, the above-mentioned one time ingestion amount can be appropriately determined. It is generally not less than 80 g, preferably not less than 100 g, not less than 110 g, not less than 115 g or not less than 120 g. When the one time ingestion amount is less than 80 g, the concentration becomes high when the necessary amount of carbohydrate and any one or more kinds of alanine, proline, and glycine are contained, and the texture and food texture may be poor. The one time ingestion amount is generally not more than 300 g, preferably not more than 250 g, not more than 200 g, not more than 150 g, not more than 140 g. When the one time ingestion amount exceeds 300 g, ingestion thereof before exercise (for example, 30 minutes to immediately before exercise) may give a burden on the gastrointestinal tract to possibly cause low exercise performance. Therefore, the range of one time ingestion amount of a jelly or a liquid is generally 80 to 300 g, preferably 100 g to 250 g, 100 g to 200 g, 100 g to 150 g.

When the form of the amino acid composition of the present invention is a jelly or a liquid, the total concentration of any one or more kinds of alanine, proline, and glycine in the amino acid composition of the present invention is generally 1 wt % to 10 wt %, preferably 1.5 wt % to 8 wt %, more preferably 2 wt % to 6 wt %, based on the total weight of the composition. When the form of the amino acid composition of the present invention is a jelly or a liquid, the total concentration of carbohydrates in the amino acid composition of the present invention is generally 10 wt % to 70 wt %, preferably 15 wt % to 60 wt %, more preferably 20 wt % to 50 wt %, based on the total weight of the composition.

The amino acid composition of the present invention may contain an amino acid other than alanine, proline, and glycine. The other amino acid is not particularly limited as long as it is a naturally occurring amino acid and other than alanine, proline, and glycine. Examples of other amino acid include valine, leucine, isoleucine, phenylalanine, tryptophan, methionine, serine, threonine, cysteine, glutamine, asparagine, tyrosine, lysine, arginine, histidine, aspartic acid, glutamic acid, and the like, which can be used for the production of foods.

When the content of the above-mentioned other amino acid is high and when the amino acid composition of the present invention is a jelly, a liquid and the like, complete dissolution of any one or more kinds of amino acids from alanine, proline, and glycine becomes difficult, an appropriate form of a jelly, a liquid and the like cannot be provided. Therefore, the total concentration of other amino acid in the amino acid composition of the present invention is preferably not more than 5 wt %, based on the solid content of the composition. In addition, absence of other amino acid is preferable. Furthermore, the amino acid composition of the present invention preferable contains alanine, proline, and glycine alone, or alanine and proline alone, or alanine and glycine alone, as the amino acid.

The amino acid composition of the present invention can also contain vitamins and minerals. Examples of the vitamins include liposoluble vitamins, for example, vitamin A such as retinol, retinal, retinoic acid, and the like, a carotenoid such as β-carotene and the like, vitamin D such as ergocalciferol, cholecalciferol, and the like, vitamin E such as α-tocopherol and the like, vitamin K such as phylloquinone, menaquinone and the like, and water-soluble vitamins, for example, vitamin B₁ such as thiamine and the like, vitamin B₂ such as riboflavin and the like, vitamin B₆ such as pyridoxine, pyridoxal, pyridoxamine, and the like, vitamin B₁₂ such as cyanocobalamin and the like, niacin such as nicotinic acid, nicotinamide, and the like, vitamin B complex such as pantothenic acid, biotin, folic acid, and the like, and vitamin C. Examples of the minerals include general minerals such as sodium, potassium, magnesium, calcium, phosphorus, iodine, iron, copper, manganese, selenium, zinc, chrome, molybdenum, and the like.

The amino acid composition of the present invention may contain other materials for food and the like or food additives, which are generally used for the production of food and drink and the like. Examples thereof include, but are not limited to, a thickener, suspending agent, dispersing agent, sweetening agent, corrigent, preservative, flavor, organic acid, gelling agent, pH adjuster, and the like.

Examples of the thickener include polymers such as dextrin, sodium alginate, alginic acid propyleneglycol ester, tragacanth powder, xanthan gum, sodium carboxymethylcellulose, hydroxypropylcellulose, polyvinyl alcohol, polyvinylpyrrolidone, and the like. Examples of the suspending agent include gum arabic, sodium alginate, sodium carboxymethylcellulose, methylcellulose, bentonite, and the like. Examples of the dispersing agent include sodium pyrophosphate, sodium polyphosphate, sodium metaphosphate, and the like. Examples of the sweetening agent include glucose, fructose, invert sugar, sorbitol, xylitol, glycerol, simple syrup, and the like. Examples of the corrigent include aspartame, saccharin, saccharin sodium, glycyrrhizic acid, monoammonium glycyrrhizinate, diammonium glycyrrhizinate, dipotassium glycyrrhizinate, disodium glycyrrhizinate, trisodium glycyrrhizinate, acesulfame potassium, mannitol, erythritol, sorbitol, xylitol, trehalose, cacao powder, and the like. Examples of the preservative include middle chain fatty acid monoglyceride, glycine, organic acid salt (e.g., sodium acetate, sodium citrate, sodium succinate, sodium fumarate), ethanol, and the like. Examples of the flavor include lemon flavor, orange flavor, grapefruit flavor, chocolate flavor, apple flavor, dl-menthol, l-menthol, and the like. Examples of the organic acid include anhydrous citric acid, citric acid, dl-malic acid, tartaric acid, d-tartaric acid, ascorbic acid, acetic acid, lactic acid, succinic acid, maleic acid, malonic acid, and the like. Examples of the gelling agent include agar, carageenan, xanthan gum, guar gum, pectin, gellan gum, locust bean gum, gum arabic, tragacanth and gelatin, and the like. Examples of the pH adjuster include citric acid hydrate or a salt thereof, disodium hydrogen phosphate anhydrous, tartaric acid or a salt thereof, sodium hydroxide, and the like.

The amino acid composition of the present invention can be produced by a general production technique for food and the like.

Ingestion of the amino acid composition of the present invention can suppress an increase of blood glucose level immediately after ingestion, as compared to ingestion of isocaloric carbohydrate. Here, the term “isocaloric carbohydrate” means a carbohydrate having the same calories as the total calories of the carbohydrates and any one or more kinds of alanine, proline, and glycine contained in the amino acid composition of the present invention, and mean the same kind of carbohydrate contained in the amino acid composition of the present invention. The blood glucose level can be measured by a method known per se, and an appropriate method can be selected according to the object. The “increase in the blood glucose level immediately after ingestion” means an increase in the blood glucose level, which occurs from ingestion to 90 minutes later, preferably 60 minutes later, more preferably 30 minutes later, in, for example, a healthy human adult, though subject to change according to the target. A method for evaluating suppression of an increase in the blood glucose level can be appropriately selected according to the object and, for example, a method of evaluation in mouse or human and the like can be mentioned. Specifically, for example, a target individual ingests the amino acid composition of the present invention, and changes in the blood glucose level (blood glucose level after a given time from ingestion minus that before ingestion, which is generally positive value) are examined before ingestion and a given time after ingestion (e.g., 15 minutes later). Similarly, an isocaloric carbohydrate is ingested and changes in the blood glucose level are examined. When changes in the blood glucose after ingestion of the amino acid composition of the present invention are lower than those after ingestion of isocaloric carbohydrate, it is evaluated that an increase in the blood glucose level was suppressed.

When the amino acid composition of the present invention is ingested, a decrease in the blood glucose level due to a prolonged exercise can be suppressed as compared to the ingestion of isocaloric carbohydrate. Here, the “isocaloric carbohydrate” is as defined above. A method for evaluating suppression of a decrease in the blood glucose level due to a prolonged exercise can be appropriately selected according to the object and, for example, a method of evaluating in mouse or human and the like can be mentioned. To be specific, for example, a target individual ingests the amino acid composition of the present invention and, after a given resting period (e.g., 15 minutes), starts exercising. After a long time of exercise (e.g., exercise for 60 minutes or longer, 90 minutes or longer, and the like), the blood glucose level is measured, and changes in the blood glucose level (blood glucose level after exercise minus blood glucose level before ingestion) are examined. Similarly, changes in the blood glucose level are examined after ingestion of isocaloric carbohydrate. When changes in the blood glucose level are maintained in higher values by ingestion of the amino acid composition of the present invention than ingestion of isocaloric carbohydrate, it can be evaluated that a decrease in the blood glucose level was suppressed. The “prolonged exercise” is an exercise for a sufficiently long time according to the age, sex, body weight of the target individual, the kind of exercise and the like and is, for example, an exercise for 30 to 180 minutes, preferably 45 to 150 minutes, more preferably 60 to 120 minutes, for a healthy human adult.

Since the amino acid composition of the present invention has the above-mentioned effect of suppressing an increase of blood glucose level immediately after ingestion and an effect of suppressing a decrease of blood glucose level due to prolonged exercise after ingestion, it is useful for energy supplementation, particularly energy supplementation before exercise for a long time.

Furthermore, the amino acid composition of the present invention can improve exercise performance. The “exercise performance” generally means an ability of a subject to achieve desired results relating to strength, speed, endurance, accuracy and the like in exercise. In the present invention, the “exercise performance” refers to an ability mainly relating to endurance.

Improvement of exercise performance can be recognized as, for example, a longer exercise time or distance and the like before reaching fatigue as compared to the control. While the control can be set as appropriate, a control who has ingested the isocaloric carbohydrate is preferably used.

A method for evaluating exercise performance is not particularly limited, and a method known in the pertinent field, for example, a method for testing and evaluation using a treadmill and a swimming pool can be used. As a specific test, an exercise load is applied to a target and the time until the target becomes unable to continue the exercise is measured. Alternatively, exercise strength (for example, running speed) is gradually increased, and the time until the target becomes unable to continue the exercise is measured. Alternatively, after a certain amount of exercise, the spontaneous motor activity of the target is measured using a commercially available spontaneous motor activity monitor and the like, and the level of fatigue of the target is evaluated. An improvement of exercise performance can also be recognized as, for example, improvements in physiological parameters such as heart rate and the like.

The amino acid composition of the present invention is useful as a composition used for improving exercise performance (e.g., endurance improvement).

The composition in the context of the present invention means a food composition similar to a pharmaceutical product, which has particular functions and aims at health maintenance and the like, such as health assistant food, food with health claims, supplement and the like, or an additive for food or a pharmaceutical product, which exerts particular action/effect.

The present invention further provides an inhibitor of an increase of blood glucose level immediately after carbohydrate ingestion, which contains any one or more kinds of alanine, proline, and glycine (hereinafter to be also referred to as an inhibitor of an increase of blood glucose level of the present invention).

This agent can suppress an increase in the blood glucose level immediately after carbohydrate ingestion. Suppression of an increase in the blood glucose level can be examined in the same manner as with the amino acid composition of the present invention by comparison with a control (for example, the same individual who ingested isocaloric carbohydrate). Here, “isocaloric carbohydrate” is a carbohydrate of the same kind as the carbohydrate to be ingested and having the same calories as the total calories of any one or more kinds of amino acids from alanine, proline, and glycine and carbohydrate to be ingested, which are contained in the agent. Here, the carbohydrate may be any saccharide as long as it can be a source of energy supplementation.

The present invention further provides an inhibitor of a decrease in the blood glucose level due to an exercise for a long time (hereinafter to be also referred to as an inhibitor of a decrease of blood glucose level of the present invention), which contains any one or more kinds of alanine, proline, and glycine.

This agent can suppress a decrease of blood glucose level due to a prolonged exercise after carbohydrate ingestion. Suppression of a decrease of blood glucose level can be examined in the same manner as with the amino acid composition of the present invention by comparison with a control (for example, the same individual who has ingested an isocaloric carbohydrate). Here, “isocaloric carbohydrate” is as defined above for an inhibitor of an increase in the blood glucose level of the present invention.

The present invention further provides an exercise performance improver containing any one or more kinds of alanine, proline, and glycine (hereinafter to be also referred to as an exercise performance improver of the present invention).

This agent can improve exercise performance. This agent is preferably ingested together with carbohydrate. Here, the carbohydrate may be any as long as it can be a source of energy supplementation. Improvement of exercise performance can be examined in the same manner as with the amino acid composition of the present invention by comparison with a control (for example, the same individual who has ingested isocaloric carbohydrate). Here, “isocaloric carbohydrate” is as defined above for an inhibitor of an increase of blood glucose level of the present invention.

An explanation relating to alanine, proline, and glycine in the inhibitor of an increase of blood glucose level of the present invention, the inhibitor of a decrease of blood glucose level, and an exercise performance improver (hereinafter to be also referred to as the agent of the present invention), the blending ratio thereof, the form of the agents and the like can refer the descriptions relating to the above-mentioned amino acid composition of the present invention.

The agent in the context of the present invention can be provided as a food composition to exert particular action effect, which is similar to a pharmaceutical product aiming at health maintenance and the like and having a particular function such as pharmaceutical product, food additive, health assistant food, food with health claims, supplement and the like.

The total amount of any one or more kinds of alanine, proline, and glycine in one dose of the agent of the present invention is the agent of the present invention is 2.5 g to 15 g, preferably 3 g to 12 g, more preferably 4 g to 10 g.

The total concentration of any one or more kinds of alanine, proline, and glycine in the agent of the present invention is generally 2 wt % to 50 wt %, preferably 3 wt % to 30 wt %, more preferably 5 wt % to 20 wt %, based on the solid content of the agent.

When the agent of the present invention is in the form of a jelly or a liquid, the total concentration of any one or more kinds of alanine, proline, and glycine in the agent of the present invention is generally 1 wt % to 10 wt %, preferably 1.5 wt % to 8 wt %, more preferably 2 wt % to 6 wt %, based on the total weight of the agent.

While the agent of the present invention does not necessarily contain carbohydrates, when it contains carbohydrate, the kind, amount, and the like thereof can be appropriately determined in the same manner as in the above-mentioned amino acid composition of the present invention.

When the agent of the present invention does not contain carbohydrates, carbohydrate may be ingested simultaneously with or separately from the ingestion of the agent of the present invention, and the order of ingestion of these is optional.

The agent of the present invention can contain an amino acid other than alanine, proline, and glycine, vitamins, minerals, food material or food additive as appropriate. As these, those used for the above-mentioned amino acid composition of the present invention can be used.

Other features of the invention will become apparent in the course of the following descriptions of exemplary embodiments which are given for illustration of the invention and are not intended to be limiting thereof.

### EXAMPLES

### Example 1

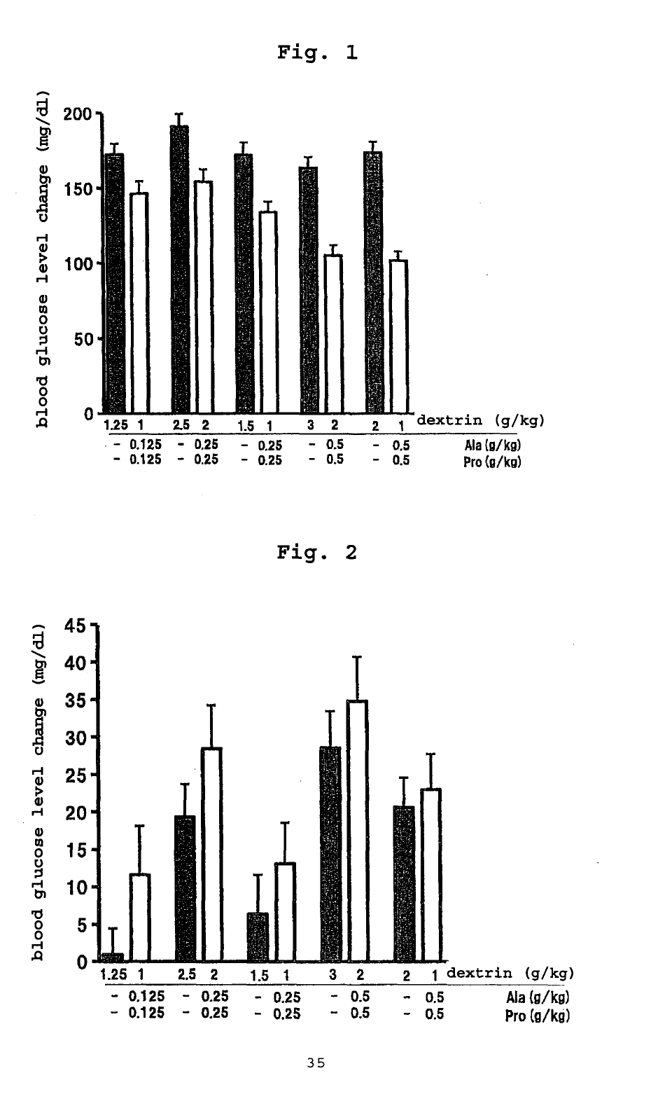

Male C57BL/6J mice (CLEA Japan, Inc.) were habituated to a running exercise using the running wheel of Tohoku Pharmaceutical University type (Nagasawa Rikagaku Kikiten), and subjected to a test. On the day of experiment, the blood glucose level of the overnight fasted mice was measured by tail vein blood sampling, and any of (1) dextrin 1.25 g/kg, (2) dextrin 1 g/kg, DL-alanine(Ala) 0.125 g/kg, L-proline(Pro) 0.125 g/kg, (3) dextrin 2.5 g/kg, (4) dextrin 2 g/kg, DL-Ala 0.25 g/kg, L-Pro 0.25 g/kg, (5) dextrin 1.5 g/kg, (6) dextrin 1 g/kg, DL-Ala 0.25 g/kg, L-Pro 0.25 g/kg, (7) dextrin 3 g/kg, (8) dextrin 2 g/kg, DL-Ala 0.5 g/kg, L-Pro 0.5 g/kg, (9) dextrin 2 g/kg, and (10) dextrin 1 g/kg, DL-Ala 0.5 g/kg, L-Pro 0.5 g/kg (18 mice/group) was administered. Each test solution was dissolved in water (10 ml/mouse body weight (kg)) and orally administered. At 15 min after the administration, the blood glucose level was measured, and a running exercise loading was started at a rate of 10.5 m/minute using the running wheel. After 90 minutes of the exercise loading, the blood glucose level was measured again. All of the blood glucose levels were measured by Glucocard G+meter (ARKRAY, Inc.). The results are shown in FIGS. 1 and 2.

FIG. 1 shows changes in the blood glucose level 15 minutes after the administration from that before administration, and FIG. 2 shows changes in the blood glucose level after 90 minutes exercise loading from that before administration. Irrespective of dose, simultaneous administration of dextrin, and Ala and Pro suppressed the increase of blood glucose level 15 minutes after the administration, and the decrease of blood glucose level after the exercise loading, as compared to an administration of an isocaloric dextrin alone. The results show that the amino acid-containing composition of the present invention is useful since it has a suppressive action on an increase in the blood glucose level and an action to maintain the blood glucose level during exercise loading.

### Example 2

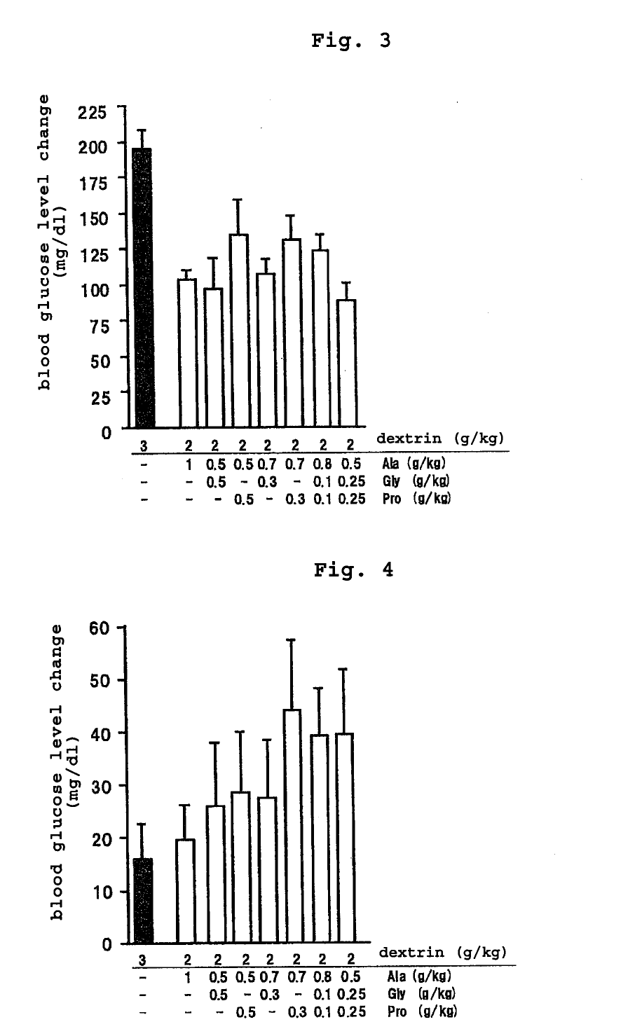

Male C57BL/6J mice (CLEA Japan, Inc.) were habituated to a running exercise using the running wheel of Tohoku Pharmaceutical University type (Nagasawa Rikagaku Kikiten), and substituted to a test. The test method is similar to that of Example 1. As the materials for administration, any of (1) dextrin 3 g/kg, (2) dextrin 2 g/kg, DL-Ala 1 g/kg, (3) dextrin 2 g/kg, DL-Ala 0.5 g/kg, glycine (Gly) 0.5 g/kg, (4) dextrin 2 g/kg, DL-Ala 0.5 g/kg, L-Pro 0.5 g/kg, (5) dextrin 2 g/kg, DL-Ala 0.7 g/kg, Gly 0.3 g/kg, (6) dextrin 2 g/kg, DL-Ala 0.7 g/kg, L-Pro 0.3 g/kg, (7) dextrin 2 g/kg, DL-Ala 0.8 g/kg, Gly 0.1 g/kg, L-Pro 0.1 g/kg, and (8) dextrin 2 g/kg, DL-Ala 0.5 g/kg, L-Gly 0.25 g/kg, L-Pro 0.25 g/kg was orally administered (6 mice/group). The results of changes in the blood glucose level are shown in FIGS. 3 and 4.

FIG. 3 shows changes in the blood glucose level 15 minutes after the administration from that before administration, and FIG. 4 shows changes in the blood glucose level after 90 minutes exercise loading from that before administration. Irrespective of dose, simultaneous administration of dextrin and, Ala alone, two kinds of Ala and Gly, two kinds of Ala and Pro, or three kinds of Ala, Gly and Pro, suppressed the increase of blood glucose level 15 minutes after the administration, and the decrease of blood glucose level after the exercise loading, as compared to an administration of an isocaloric dextrin alone. The results show that the amino acid-containing composition of the present invention is useful since it has a suppressive action on the increase of blood glucose level and an action to maintain the blood glucose level during exercise loading.

### Example 3

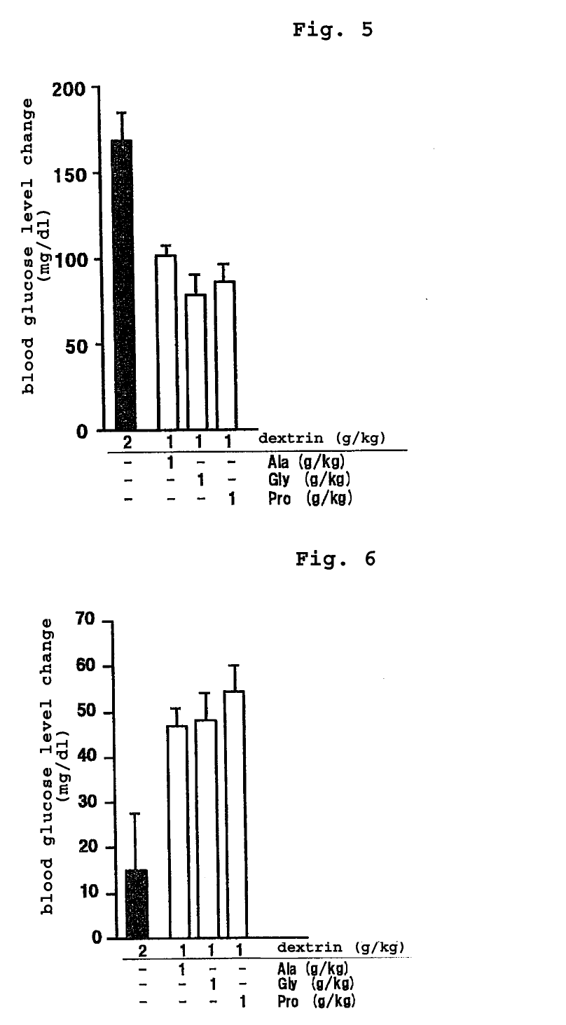

Male C57BL/6J mice (CLEA Japan, Inc.) were habituated to a running exercise using the running wheel of Tohoku Pharmaceutical University type (Nagasawa Rikagaku Kikiten), and subjected to a test. The test method is similar to that of Example 1. As the materials for administration, any of (1) dextrin 2 g/kg, (2) dextrin 1 g/kg, DL-Ala 1 g/kg, (3) dextrin 1 g/kg, Gly 1 g/kg, and (4) dextrin 1 g/kg, L-Pro 1 g/kg was orally administered (6 mice/group). The results of changes in the blood glucose level are shown in FIGS. 5 and 6.

FIG. 5 shows changes in the blood glucose level 15 minutes after the administration from that before administration, and FIG. 6 shows changes in the blood glucose level after 90 minutes exercise loading from that before administration. As compared to a dextrin administration alone, when a mixture of dextrin, and any of amino acid from Ala, Gly and Pro (equal calories to dextrin alone) was administered, the increase of blood glucose level 15 minutes after the administration, and the decrease of blood glucose level after the exercise loading were suppressed. The results show that the amino acid-containing composition of the present invention is useful since it has a suppressive action on an increase in the blood glucose level and an action to maintain the blood glucose level during exercise loading.

### EXAMPLE 4

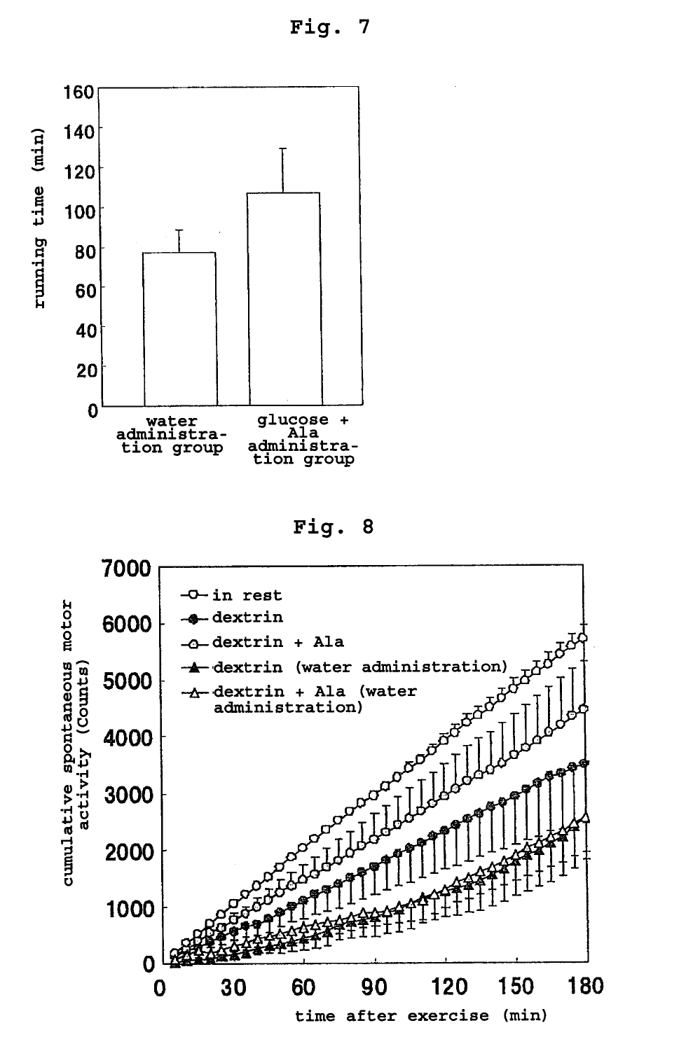

Male C57BL/6J mice were habituated to a running exercise using a treadmill (Arco System), and subjected to a test. The mice were fasted overnight, and orally administered (6 mice/group) (1) water, or (2) glucose 1 g/kg and L-alanine (Ala) 1 g/kg, and from 15 minutes after the administration, a running exercise was started using the treadmill at a rate of 22 m/minute. At the time point when the mouse could not escape from the electric grid and 5 seconds passed therefrom, the mouse was evaluated as exhausted, and the running time until exhaustion of the mouse was measured. The results are shown in FIG. 7.

FIG. 7 shows the running time until exhaustion. As compared to the water administration group, the group simultaneously administered with glucose and Ala as the energy source (glucose+Ala administration group) showed an prolonged running time, and it was clarified that energy supplementation does not decrease exercise performance but improves more than water administration.

### Example 5

Male C57BL/6J mice were habituated to a running exercise using a treadmill, and subjected to a test. The mice were fasted overnight, and orally administered (1) dextrin 2 g/kg, or (2) dextrin 1 g/kg and L-alanine (Ala) 1 g/kg. From 15 minutes after the administration, a 90 minutes running exercise was loaded at a rate of 26 m/minute using the treadmill. After exercise loading, the mice were transferred into a cage, and the motor activity was measured using a spontaneous motor activity monitor (NS-AS01: Neuroscience Inc.) for 3 hours (dextrin and dextrin+Ala in FIG. 8). As a control, the mice administered the above-mentioned (1) or (2) were administered water instead of (1) or (2), and the spontaneous motor activity was measured in the same manner (dextrin (water administration) and dextrin+Ala (water administration) in FIG. 8). In addition, the spontaneous motor activity of mice in a resting state after fasting overnight and free of the exercise loading was measured for 3 hours in the same manner (in rest in FIG. 8).

FIG. 8 shows the results of the spontaneous motor activity (6-12 mice/group). As compared to the water administration, the motor activity increased by the administration of dextrin to be the energy. Simultaneous administration of dextrin and Ala was observed increase in the motor activity more than that by the administration of dextrin alone, even though the energy (calories) was equivalent to dextrin alone. The results have clarified that simultaneous administration of dextrin and Ala reduces fatigue caused by running exercise loading more than the administration of dextrin alone, and increases the spontaneous motor activity.

### Example 6

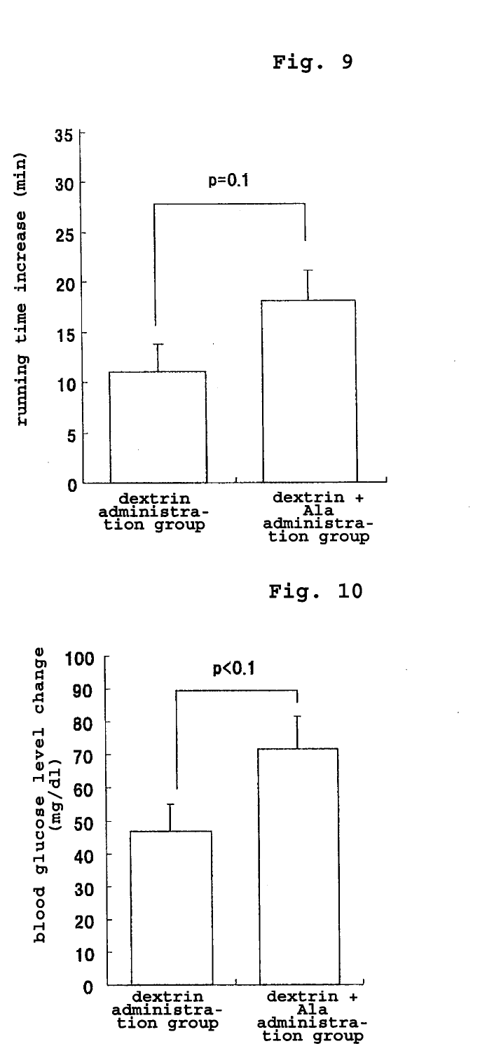

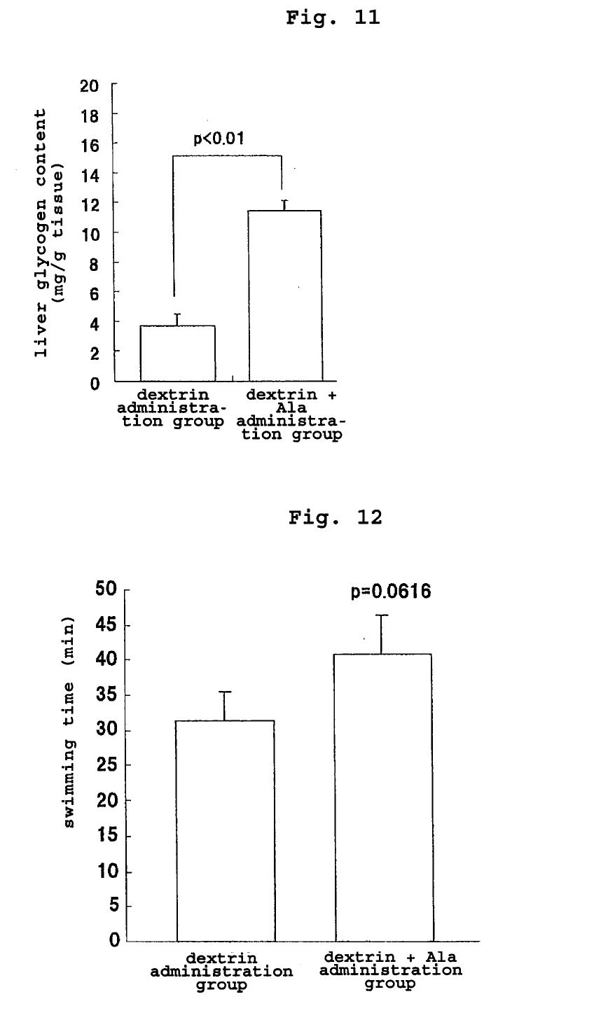

Male C57BL/6J mice were habituated to a running exercise using a treadmill, and subjected to a test. The mice were fasted overnight, and loaded with a 60 minutes running exercise at 14 m/minute during non-administration. After 60 minutes, the speed was increased every 5 minutes by 2 m/minute up to 36 m/minute, and the running time until exhaustion was measured. After 1 week from the measurement, the mouse was again fasted overnight, orally administered (9 mice/group) (1) dextrin 2 g/kg, (2) dextrin 1 g/kg and L-alanine (Ala) 1 g/kg, and studied in the same manner as in non-administration. The running exercise loading at a rate of 14 m/minute was started from 15 minutes after the oral administration. An increase in the running time from that of each non-administration is shown in FIG. 9. FIG. 10 shows changes in the blood glucose level after 60 minutes running at 14 m/minute from immediately before administration, and FIG. 11 shows liver glycogen content after 60 minutes running at 14 m/minute.

As shown in FIG. 9, the running time from each non-administration increased by simultaneous administration of dextrin and Ala as compared to administration of an equal energy (calories) amount of dextrin alone. As shown in FIGS. 10 and 11, the dextrin+Ala administration group also showed high values of changes in the blood glucose level and liver glycogen content after the same exercise loading. From the above, it has been shown that simultaneous administration of dextrin and Ala suppresses the decrease of blood glucose level due to exercise loading, and maintains liver glycogen content. Furthermore, it has been clarified that simultaneous administration of dextrin and Ala makes it possible to endure an increase in the exercise loading for a long time as compared to administration of dextrin alone.

### Example 7

Male C57BL/6J mice were habituated to a swimming exercise by the flowing water pool of Kyoto University Matsumoto type motor activity measurement reformed by Ishihara (Anitec), and subjected to a test. The mice were fasted overnight, and orally administered (39 mice/group) (1) dextrin 2 g/kg, or (2) dextrin 1 g/kg and L-alanine (Ala) 1 g/kg. After 15 minutes from the administration, the swimming exercise was started at a flow rate of 11 m/minute. The time point when the tip of the nose of mouse sank under water and 7 seconds passed therefrom was taken as exhaustion, and the swimming time until exhaustion of mouse was measured. The results are shown in FIG. 12.

As shown in FIG. 12, it was clarified that the swimming time is prolonged by about 30% by simultaneous administration of Ala as compared to the administration of dextrin alone, even though an equal energy (calories) amount was orally administered.

### Example 8

Each component was added to ion exchange water and, after dissolution by heating, filled in a plugged aluminum pouch to produce an amino acid jelly composition (100 g) having the following composition. Ion exchange water 49.00 g, DL-alanine 4.50 g, L-proline 0.50 g, dextrin 44.50 g, acidulant (citric acid and sodium citrate) 0.80 g, gelling agent (agar) 0.60 g and flavor 0.10 g.

### Example 9

Each component was added to ion exchange water and, after dissolution by heating, filled in a plugged aluminum pouch to produce an amino acid jelly composition (130 g) having the following composition. Ion exchange water 78.85 g (60.65 wt %), DL-alanine 4.50 g (3.46 wt %), L-proline 0.50 g (0.38 wt %), dextrin 44.50 g (34.23 wt %), acidulant (citric acid and sodium citrate) 0.74 g (0.57 wt %), gelling agent (agar) 0.78 g (0.6 wt %) and flavor 0.13 g (0.10 wt %).

### Example 10

Each component was added to ion exchange water and, after dissolution by heating, filled in a plugged aluminum pouch to produce an amino acid gel composition (100 g) having the following composition. Ion exchange water 48.85 g, DL-alanine 4.50 g, L-proline 0.50 g, dextrin 44.50 g, granulated sugar 0.75 g, acidulant (citric acid and sodium citrate) 0.80 g and flavor 0.10 g.

### Example 11

Male C57BL/6J mice (CLEA Japan, Inc.) were habituated to a running exercise using the running wheel of Tohoku Pharmaceutical University type (Nagasawa Rikagaku Kikiten), and subjected to a test. On the day of experiment, the blood glucose level of the overnight fasted mice was measured by tail vein blood sampling, and any of (1) dextrin 1.25 g/kg, or (2) dextrin 1 g/kg, DL-Ala 0.225 g/kg and L-Pro 0.025 g/kg was orally administered (18 mice/group).

Each test solution was dissolved in water (10 ml/mouse body weight (kg)) and orally administered. At 15 minutes after the administration, the blood glucose level was measured, and a running exercise loading was started at a rate of 10.5 m/minute using the running wheel. The blood glucose level was measured 30 minutes, 65 minutes, 100 minutes, 135 minutes, and 170 minutes from the start of exercise. All of the blood glucose levels were measured by Glucocard G+meter (ARKRAY, Inc.).

FIG. 13 shows the blood glucose levels before administration, 15 minutes after the administration (0 minutes after start of exercise) and after the start of the exercise.

As compared to dextrin alone, a mixture of dextrin, Ala and Pro significantly suppressed the increase of blood glucose level 15 minutes after the administration, and the decrease of blood glucose level after 100 minutes exercise loading. The results show that the amino acid-containing composition of the present invention is useful since it has a suppressive action on an increase in the blood glucose level and an action to maintain the blood glucose level during exercise loading.

### Example 12

Male C57BL/6J mice (CLEA Japan, Inc.) were habituated to a swimming exercise by the flowing water pool of Kyoto University Matsumoto type motor activity measurement reformed by Ishihara (Anitec), and subjected to a test. The mice were fasted from 3 hours before the start of the test, and orally administered (18 mice/group) (1) dextrin 2 g/kg, or (2) dextrin 1 g/kg, DL-Ala 0.9 g/kg and L-Pro 0.1 g/kg. After 15 minutes from the administration, the swimming exercise was started at a flow rate of 11 m/minute. The time point when the tip of the nose of mouse sank under water and 7 seconds passed therefrom was taken as exhaustion, and the swimming time until exhaustion of mouse was measured.

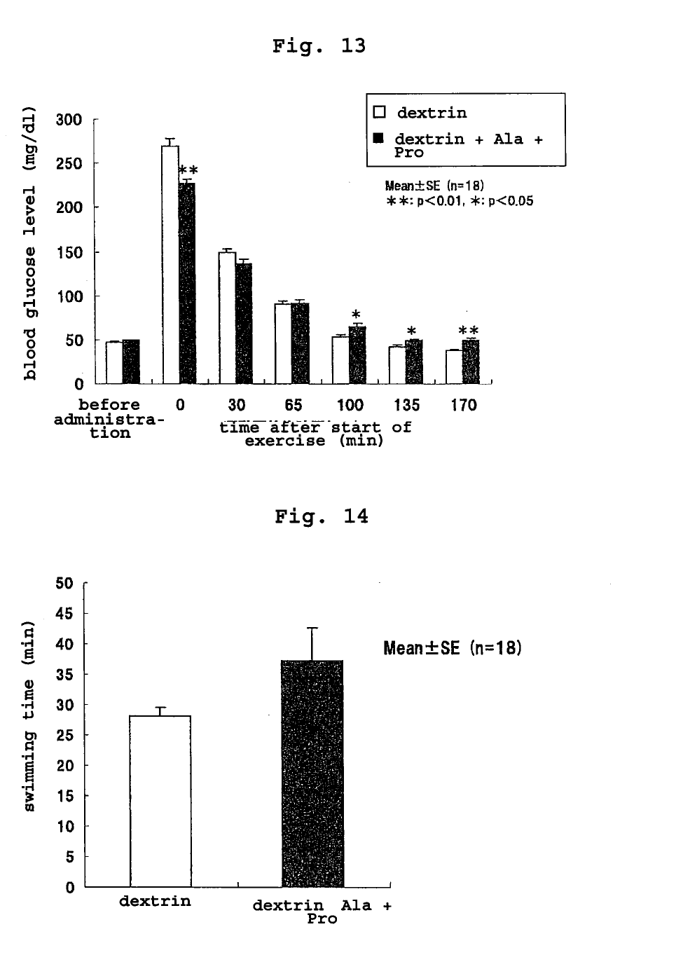

As shown in FIG. 14, it was clarified that the administration of a mixture of dextrin, Ala and Pro extends the swimming time as compared to the administration of an equal energy (calories) amount of dextrin alone.

### Example 13

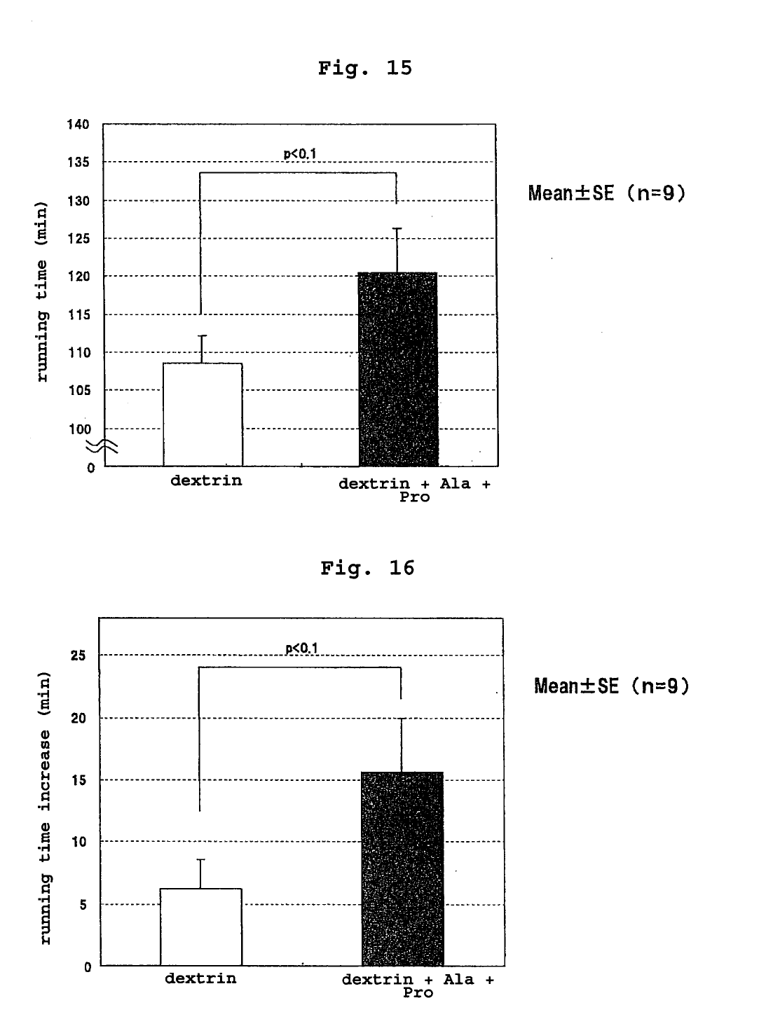

Male C57BL/6J mice (CLEA Japan, Inc.) were habituated to a running exercise using a treadmill, and subjected to a test. The mice were fasted overnight, orally administered with distilled water, and loaded with a 60 minutes running exercise at a rate of 14 m/minute after 15 minutes from the administration. After 60 minutes, the speed was increased every 5 minutes by 2 m/minute up to 36 m/minute, the running time until exhaustion was measured, and the exercise capacity of each mouse was evaluated. One week later, the mice were divided into two groups showing no difference in the exercise capacity (running time), fasted again overnight, orally administered (9 mice/group) (1) dextrin 2 g/kg, or (2) dextrin 1 g/kg, DL-Ala 0.9 g/kg and L-Pro 0.1 g/kg, and studied in the same manner as in the administration of distilled water. FIG. 15 shows the running time, and FIG. 16 shows an increase in the running time of each mouse, which is obtained by subtracting the running time with administration of distilled water.

As shown in FIGS. 15 and 16, the administration of the mixture of dextrin, DL-Ala and L-Pro increased the running time and an increased amount of the running time as compared to the administration of an equal energy (calories) amount of dextrin alone. From the above, it was clarified that the administration of the mixture of dextrin, Ala and Pro makes it possible to endure an increase in the exercise loading for a long time as compared to the administration of dextrin alone.

As described above, it is clear that the amino acid composition provided by the present invention suppresses the increase of blood glucose level immediately after ingestion, suppresses the decrease of blood glucose level after a long time exercise, and improves exercise performance by ingestion before the exercise.

### INDUSTRIAL APPLICABILITY

The present invention provides foods and drinks, and pharmaceutical products for energy supplementation, which are useful for those who exercise, particularly general public and athletes who exercise for a long time.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An amino acid composition, comprising:
(a) at least one carbohydrate; and
(b) one or more amino acids selected from the group consisting of alanine, proline, and glycine.

2. An amino acid composition according to claim 1, wherein a total concentration of said one or more amino acids selected from the group consisting of alanine, proline, and glycine is 2 wt % to 50 wt %, based in the solid content of said composition.

3. An amino acid composition according to claim 1, wherein a total concentration of said at least one carbohydrate is 10 wt % to 90 wt %, based in the solid content of said composition.

4. An amino acid composition according to claim 1, which is in the form of a package of a unit ingestion amount of 2.5 g to 15 g in total of said one or more amino acids selected from the group consisting of alanine, proline, and glycine.

5. An amino acid composition according to claim 1, comprising:
(b′) alanine; and
(b″) proline and/or glycine.

6. An amino acid composition according to claim 5, wherein the weight ratio of (b′) alanine to (b″) proline and/or glycine is 1:0.01 to 1.

7. An amino acid composition according to claim 1, which is in the form of a jelly or a liquid.

8. An amino acid composition according to claim 1, which suppresses an increase of blood glucose level immediately after ingestion as compared to ingestion of isocaloric carbohydrate.

9. An amino acid composition according to claim 1, which suppresses a decrease of blood glucose level due to prolonged exercise after ingestion as compared to ingestion of isocaloric carbohydrate.

10. An amino acid composition according to claim 1, which is used for improving exercise performance.

11. An amino acid composition according to claim 1, which is free of other amino acids.

12. An inhibitor of an increase of blood glucose level immediately after carbohydrate ingestion, comprising one or more amino acids selected from the group consisting of alanine, proline, and glycine.

13. An inhibitor of a decrease of blood glucose level due to a prolonged exercise, comprising any one or more kinds of alanine, proline and glycine.

14. An exercise performance improver, comprising one or more amino acids selected from the group consisting of alanine, proline, and glycine.

15. A method of suppressing an increase of blood glucose level immediately after carbohydrate ingestion, comprising administering a composition comprising one or more amino acids selected from the group consisting of alanine, proline, and glycine, to a subject in need thereof.

16. A method of suppressing a decrease of blood glucose level due to prolonged exercise, comprising administering a composition comprising one or more amino acids selected from the group consisting of alanine, proline, and glycine, to a subject in need thereof.

17. A method of improving exercise performance, comprising administering a composition comprising one or more amino acids selected from the group consisting of alanine, proline, and glycine, to a subject in need thereof.
